(19) United States
(12) Patent Application Publication
(10) Pub. No.: US 2018/0184969 A1
(43) Pub. Date: Jul. 5, 2018

(54) SMART EYESHIELD

(71) Applicants: BOE TECHNOLOGY GROUP CO., LTD., Beijing (CN); BEIJING BOE OPTOELECTRONICS TECHNOLOGY CO., LTD., Beijing (CN)

(72) Inventors: Detao ZHAO, Beijing (CN); Huiying LI, Beijing (CN); Hanqing LIU, Beijing (CN); Bin LI, Beijing (CN); Pengju ZHANG, Beijing (CN)

(73) Assignees: BOE TECHNOLOGY GROUP CO., LTD., Beijing (CN); BEIJING BOE OPTOELECTRONICS TECHNOLOGY CO., LTD., Beijing (CN)

(21) Appl. No.: 15/526,980

(22) PCT Filed: Aug. 5, 2016
(86) PCT No.: PCT/CN2016/093533

§ 371 Date: May 15, 2017

(30) Foreign Application Priority Data
Apr. 7, 2016 (CN) 201610212096.7

(51) Int. Cl. A61B 5/00

(52) U.S. Cl. CPC A61B 5/4809; A61F 9/04; A61B 5/4812

(57) ABSTRACT

The present disclosure relates to the technical field of intelligent wearable device, and in particular to a smart eyeshield, in an attempt to improve sleep qualities of sleeping persons. The smart eyeshield comprises: a sleep monitoring system for tracking and monitoring a sleep state of a user; and a gentle wake-up system for wakening the user in a gentle manner depending on a feedback provided by the sleep monitoring system.

### FIELD

The present disclosure relates to the technical field of intelligent wearable device, and in particular to a smart eyeshield.

### BACKGROUND ART

As the rhythm of life is getting more and more rapid, how to effectively ensure people's sleep quality is becoming increasingly important. Conventionally, when sleeping persons are wakened by functions such as a sudden sound or vibration initiated by devices such as an alarm clock, adverse effects are often exerted on their heart rates, in vivo hormone levels and the like. This gives rise to problems of bad sleep quality such as physical discomfort and spiritual fatigue. A current intelligent wearable device, for example, a smart bracelet capable of monitoring activities like heartbeat and respiration, usually only has a function of monitoring a sleep quality and issuing a prompt, but fails to beneficially adjust the sleep quality at the same time by taking advantage of the monitoring results. Therefore, with such wearable devices, sleeping persons can only monitor and collect information about sleep states, but as for how to improve the sleep quality, they can do nothing but resort to other measures, for example, by wearing a light-shielding eyeshield, by avoiding intensive exercise before sleep, by making a regular sleep time table and so on.

### SUMMARY

To this end, the present disclosure provides a smart eyeshield, in an attempt to solve problems such as physical discomfort and spiritual fatigue of sleeping persons as mentioned above, thereby improving their sleep quality.

According to embodiments of the present disclosure, a smart eyeshield is provided, comprising: a sleep monitoring system for tracking and monitoring a sleep state of a user; and a gentle wake-up system for wakening the user in a gentle manner depending on a feedback provided by the sleep monitoring system.

In the smart eyeshield provided by embodiments of the present disclosure, firstly a sleep state of the user is tracked and monitored by the sleep monitoring system, and then a wake-up service is provided to the user by the gentle wake-up system depending on specific monitoring results. In this way, the user can be wakened in a gentle manner. This helps to avoid adverse effects exerted on the heart rates of the use for example by a sudden sound or vibration sent out by an alarm clock.

In a specific embodiment, the smart eyeshield further comprises: a massage system for providing a massage to the user based on the feedback provided by the sleep monitoring system. With such a massage function of the massage system, beneficial effects on the user such as relaxation of nerves and improvement of fatigue can be achieved.

In a specific embodiment, the smart eyeshield further comprises a body and a fastener. Specifically, in a wearing state of the smart eyeshield, the body covers the user's eyes and the fastener surrounds the user's head. Besides, the sleep monitoring system, the gentle wake-up system and the massage system are arranged within the body. According to this specific embodiment, the fastener is used for securing the smart eyeshield to the user's head when in a wearing state, and the body is used for accommodating various internal components according to embodiments of the present disclosure, so as to achieve beneficial effects such as gentle wake-up, relaxation of nerves and improvement of fatigue.

Furthermore, the body comprises an external layer, an intermediate layer and an internal layer. Specifically, in a wearing state of the smart eyeshield, the external layer is exposed to outside, the internal layer is in contact with the user, and the intermediate layer is sandwiched between the external layer and the internal layer. By forming the body into a layered structure, subsequent suitable distribution of various internal components in corresponding layers is improved. This helps to facilitate manufacture, assembling or the like of the smart eyeshield.

In a specific embodiment, the sleep monitoring system comprises: an eyeball movement catcher and a brain vasculature monitor. Exemplarily, the eyeball movement catcher and the brain vasculature monitor are arranged in the internal layer of the body. Specifically, the eyeball movement catcher comprises one or more sensors for recording a rotation frequency of eyeballs. Besides, the brain vasculature monitor comprises one or more pulse sensors for recording a beating frequency of pulses. According to this specific embodiment, by tracking and monitoring eyeballs and brain vasculature, information about various sleep states of the user, such as depth, quality and duration, can be easily obtained. This helps to achieve good monitoring of sleep quality and facilitate subsequent wake-up and massage of the user, thereby improving the sleep quality.

In another specific embodiment, the gentle wake-up system comprises: a light emitting module; a receiving module coupled to the sleep monitoring system and used for receiving a feedback provided by the sleep monitoring system; a switch module coupled to the receiving module and used for controlling the light emitting module to be switched on or off based on the feedback received by the receiving module; and a brightness dimming module coupled to the receiving module and used for adjusting brightness of light emitted by the light emitting module based on the feedback received by the receiving module. Exemplarily, the receiving module, the switch module and the brightness dimming module are arranged in the external layer of the body. Besides, the light emitting module is arranged in the intermediate layer of the body. Additionally, the internal layer of the body is made of a light transmissive material. In short, the gentle wake-up system herein is a system that achieves a gentle wake-up function in an optical manner. According to this specific embodiment, the user can be wakened in a gentle manner via changes of light inside the eyeshield, for example, by varying the brightness of light from dark to bright. As compared with a conventional wake-up device such as an alarm clock, embodiment of the present disclosure avoids wakening the user by a sudden sound or vibration. Thus, adverse effects on heart rates, in vivo hormone levels and the like are reduced. As an example, the light emitting module can be a light emitting diode (LED). Obviously, those skilled in the art can foresee other equivalent substitutable light emitting modules, such as an organic light emitting diode (OLED).

In yet another specific embodiment, the gentle wake-up system comprises: a sound module; a receiving module coupled to the sleep monitoring system and used for receiving a feedback provided by the sleep monitoring system; a switch module coupled to the receiving module and used for controlling the sound module to be switched on or off based on the feedback received by the receiving module; and a volume tuning module coupled to the receiving module and used for adjusting volume of the sound module based on the feedback received by the receiving module. Exemplarily, the receiving module, the switch module and the volume tuning module are arranged in the external layer of the body. Besides, the sound module is arranged in the intermediate layer of the body. In short, the gentle wake-up system herein is a system that achieves a gentle wake-up function in an acoustic manner. Similar to the situation as described above in an optical manner, in this specific embodiment, the user can be wakened in a gentle manner by timely adjusting the volume of sound around the eyeshield based on the feedback results. This helps to avoid in a same way the adverse effects exerted by a sudden wake-up on the user's heart rates, in vivo hormone levels and the like.

In other specific embodiments, the massage system comprises a microwave massage system for giving massage to the user's eyes via a microwave massage function. Exemplarily, the microwave massage system is arranged in the intermediate layer of the body. Specifically, the microwave massage system comprises one or more microwave generators for giving microwave-massage to acupuncture points of the user's eyes. According to these specific solutions, by giving massage, specifically microwave-massage, to the user's eyes based on the monitoring results, beneficial effects such as relaxation of nerves and improvement of eye fatigue can be achieved.

In further specific embodiments, the smart eyeshield further comprises: a communication module coupled to the sleep monitoring system and used for providing to an external device information about sleep states monitored by the sleep monitoring system. Exemplarily, the communication module is arranged in the external layer of the body. Furthermore, the communication module provides the information about sleep states to the external device via a Bluetooth module or a wireless module. Exemplarily, the Bluetooth module and the wireless module are arranged in the external layer of the body. With the above communication module, various information about the sleep quality collected by the sleep monitoring system is timely transmitted to an external device such as a smart phone or a tablet computer. This helps to facilitate establishment of a sleep quality log for the user. For the user or furthermore for medical care personnel, such a sleep quality log not only enables an intuitional understanding of an evolutionary trend of the sleep state with time, but also provides a preventive measure which is effective against poor sleep quality.

### BRIEF DESCRIPTION OF DRAWINGS

These and other aspects and advantages of the present disclosure will become obvious in the detailed description below with reference to the drawings. In the drawings:

FIG. 1 is a schematic functional block diagram of a smart eyeshield according to an embodiment of the present disclosure;

FIG. 2 is a schematic structure view of a smart eyeshield according to an embodiment of the present disclosure;

FIG. 3 is a further schematic structure view of a smart eyeshield according to an embodiment of the present disclosure;

FIG. 4 is a schematic functional block diagram of a gentle wake-up system in a smart eyeshield according to an embodiment of the present disclosure; and

FIG. 5 is a schematic functional block diagram of a massage system in a smart eyeshield according to an embodiment of the present disclosure.

### DETAILED DESCRIPTION OF EMBODIMENTS

Now, the smart eyeshield provided in embodiments of the present disclosure will be described below in detail in a more comprehensive manner with reference to the appended drawings. In should be noted that the smart eyeshield can be embodied in many different ways and the protection scope should not be construed as being limited to those specific embodiments illustrated herein. On the contrary, these embodiments are merely provided for thoroughness and completeness, and aim to convey the protection scope of the present disclosure to those skilled in the art clearly and completely. The same reference signs are used for indicating the same items throughout.

Firstly, referring to FIG. 1, a schematic functional block diagram of a smart eyeshield 10 according to an embodiment of the present disclosure is shown. The smart eyeshield 10 can comprise: a sleep monitoring system 11 for tracking and monitoring a sleep state of a user; and a gentle wake-up system 12 for wakening the user in a gentle manner depending on a feedback provided by the sleep monitoring system. In such a smart eyeshield 10, firstly a sleep state of the user is tracked and monitored by the sleep monitoring system 11, and then a wake-up service is provided to the user by the gentle wake-up system 12 depending on specific monitoring results. Thereby, the user can be wakened in a gentle manner via the gentle wake-up system 12, where appropriate, for example when he/she needs to be wakened. In this way, problems such as spiritual fatigue caused by a sudden wake-up can be avoided. Furthermore, the smart eyeshield 10 can further comprise a massage system 13 for providing a massage to the user based on the feedback provided by the sleep monitoring system. In a similar way, beneficial effects such as relaxation of nerves and improvement of fatigue can be achieved via the massage system 13, where appropriate, for example when the monitoring results indicate that the sleep quality of the user is poor.

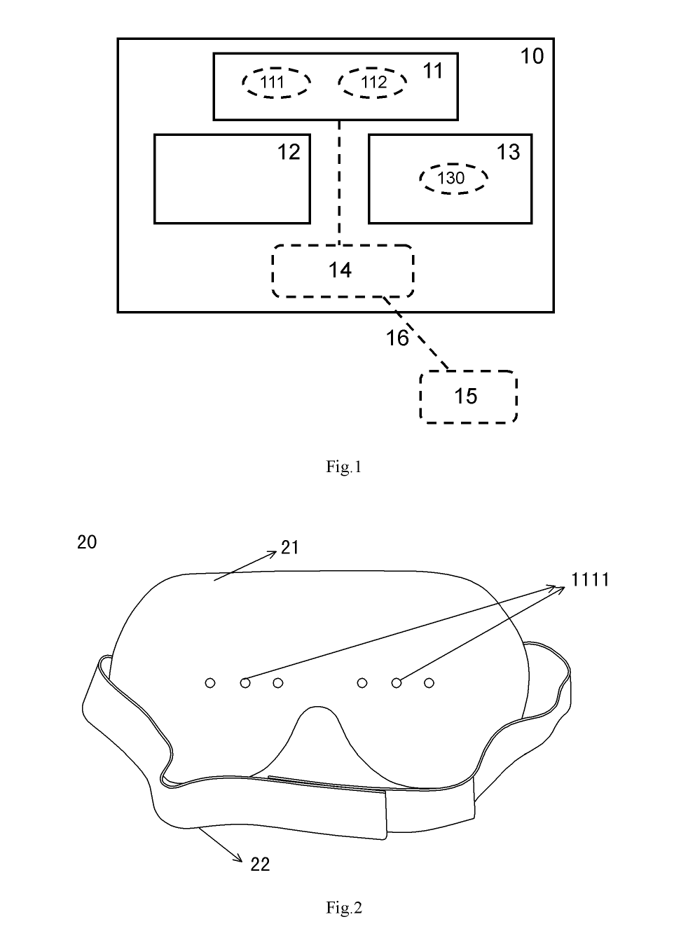

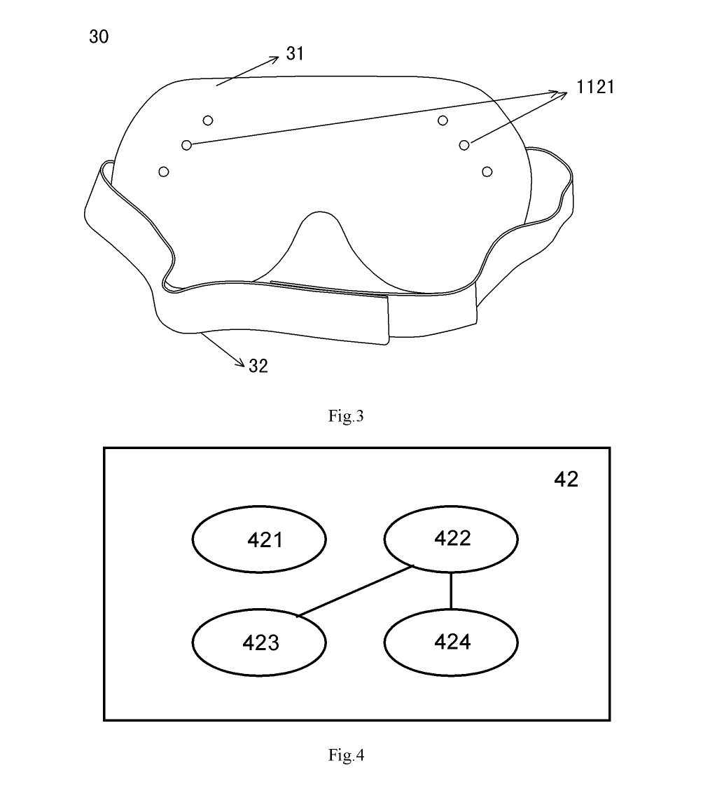

In a specific embodiment, referring to FIGS. 2 and 3, the smart eyeshield 20, 30 further comprises a body 21, 31 and a fastener 22, 32. Specifically, in a wearing state of the smart eyeshield 20, 30, the body 21, 31 covers the user's eyes and the fastener 22, 32 surrounds the user's head. Besides, the sleep monitoring system 11, the gentle wake-up system 12 and the massage system 13 are arranged within the body 21, 31. According to this specific embodiment, the fastener 22, 32 is used for securing the smart eyeshield 20, 30 to the user's head when in a wearing state, and the body 21, 31 is used for accommodating various internal components 11, 12, 13 according to embodiments of the present disclosure, so as to achieve beneficial effects such as gentle wake-up, relaxation of nerves and improvement of fatigue.

Furthermore, the body 21, 31 comprises an external layer, an intermediate layer and an internal layer (not shown). Specifically, in the wearing state of the smart eyeshield 20, 30, the external layer is exposed to outside, the internal layer is in contact with the user, and the intermediate layer is sandwiched between the external layer and the internal layer. By shaping the body into a layered structure, subsequent suitable distribution of various internal components 11, 12, 13 in corresponding layers is improved. This helps to facilitate manufacture, assembling or the like of the smart eyeshield 20, 30.

In a specific embodiment, the sleep monitoring system 11 can comprise: an eyeball movement catcher 111 and a brain vasculature monitor 112, specifically as shown in FIG. 1. Exemplarily, the eyeball movement catcher 111 and the brain vasculature monitor 112 are arranged in the internal layer (for example, the layer facing an observer in FIGS. 2 and 3) of the body 21, 31. Specifically, the eyeball movement catcher 111 can comprise one or more sensors 1111 for recording a rotation frequency of eyeballs (FIG. 2). Referring to FIG. 2, a schematic structure view of the smart eyeshield 10 according to an embodiment of the present disclosure is shown, wherein exemplary orientations of sensors 1111 in the smart eyeshield 10 are indicated by circles. Of course, the present disclosure is not limited thereto. Besides, the brain vasculature monitor 112 can comprise one or more pulse sensors 1121 for recording a beating frequency of pulses (FIG. 3). Referring to FIG. 3, a further schematic structure view of the smart eyeshield 10 according to an embodiment of the present disclosure is shown, wherein exemplary orientations of pulse sensors 1121 in the smart eyeshield 10 are indicated by circles. Again, the present disclosure is not limited thereto. According to this specific embodiment, by tracking and monitoring eyeballs and brain vasculature, information about various sleep states of the user, such as depth, quality and duration, can be easily obtained. This helps to achieve good monitoring of sleep quality and facilitate the subsequent wake-up and massage of the user, thereby improving the sleep quality.

Obviously, as can be easily understood by those skilled in the art, the sleep monitoring system 11 can further be implemented in any other suitable way, as long as it can track and monitor sleep states of the user. Exemplarily, the sleep monitoring system 11 can comprise a heartbeat monitor, a pulse monitor, a respiration monitor and so on. Besides, orientations of the sleep monitoring components as mentioned above are not limited only to those specifically described in the embodiments of the present disclosure or listed in the drawings. On the contrary, having benefits from teaching of the present disclosure, those skilled in the art can easily design other equivalent positional distributions, which should all fall within the protection scope of the present disclosure.

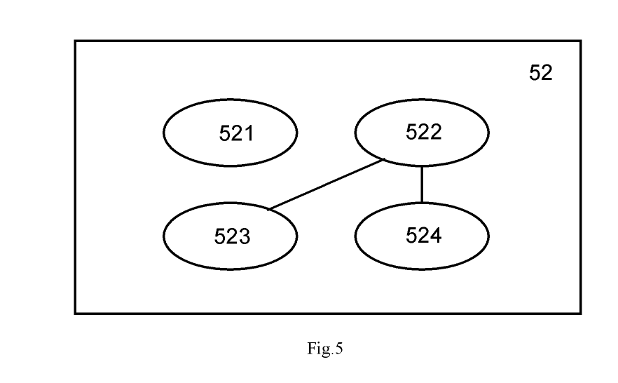

Specifically referring to FIG. 4, a specific implementation of the gentle wake-up system 42 is shown. In this specific implementation solution, the gentle wake-up system 42 can comprise: a light emitting module 421; a receiving module 422 coupled to the sleep monitoring system 11 and used for receiving a feedback provided by the sleep monitoring system 11; a switch module 423 coupled to the receiving module 422 and used for controlling the light emitting module 421 to be switched on or off based on the feedback received by the receiving module 422; and a brightness dimming module 424 coupled to the receiving module 422 and used for adjusting brightness of light emitted by the light emitting module 421 based on the feedback received by the receiving module 422. Exemplarily, the receiving module 422, the switch module 423 and the brightness dimming module 424 are arranged in the external layer (for example, a layer opposite to the layer facing the observer in FIGS. 2 and 3) of the body 21, 31. Besides, the light emitting module 421 is arranged in the intermediate layer (for example, a layer sandwiched between the internal layer and the external layer in FIGS. 2 and 3) of the body 21, 31. Furthermore, the internal layer (for example, a layer facing the observer in FIGS. 2 and 3) of the body 21, 31 is made of a light transmissive material. In short, the gentle wake-up system herein 42 is a system that achieves a gentle wake-up function in an optical manner. According to this specific embodiment, the user can be wakened in a gentle manner via changes of light inside the eyeshield 10, for example, by varying the brightness of light from dark to bright. As compared with a conventional wake-up device such as an alarm clock, embodiments of the present disclosure avoid wakening the user by a sudden sound or vibration. Thus, adverse effects on heart rates, in vivo hormone levels and the like will be reduced. As an example, the light emitting module 421 can be a light emitting diode (LED). Obviously, those skilled in the art can foresee other equivalent substitutable light emitting modules, such as an organic light emitting diode (OLED).

Specifically, the smart eyeshield 10 can store in advance a preset image. When monitoring results of the sleep monitoring system 11 indicate that the user needs to be wakened, the gentle wake-up system 42 can control, based on the monitoring results received by the receiving module 422, the light emitting module 421 to be switched on via the switch module 423, so as to display the preset image to the user. After that, the brightness dimming module 424 can further slowly adjust brightness of the image based on the monitoring results received by the receiving module 422, so as to vary the image gradually from dark to bright. Thereby, the user is wakened in a comparatively gentle manner via slow changes in the brightness of the image.

Further referring to FIG. 5, another specific implementation of the gentle wake-up system 52 is shown. In this specific implementation solution, the gentle wake-up system 52 can comprise: a sound module 521; a receiving module 522 coupled to the sleep monitoring system 11 and used for receiving a feedback provided by the sleep monitoring system 11; a switch module 523 coupled to the receiving module 522 and used for controlling the sound module 521 to be switched on or off based on the feedback received by the receiving module 522; and a volume tuning module 524 coupled to the receiving module 522 and used for adjusting volume of the sound module 521 based on the feedback received by the receiving module 522. Exemplarily, the receiving module 522, the switch module 523 and the volume tuning module 524 are arranged in the external layer of the body 21, 31 (for example, a layer opposite to the layer facing the observer in FIGS. 2, 3). Besides, the sound module 521 is arranged in the intermediate layer of the body 21, 31 (for example, a layer sandwiched between the internal layer and the external layer in FIGS. 2, 3). In short, the gentle wake-up system 52 herein is a system that achieves a gentle wake-up function in an acoustic manner. Similar to the situation as described above in an optical manner, in this embodiment, the user can be wakened in a gentle manner by timely adjusting the volume of sound around the eyeshield 10 based on the feedback results.

Specifically, the smart eyeshield 10 can store in advance a preset sound. When monitoring results of the sleep monitoring system 11 indicate that the user needs to be wakened, the gentle wake-up system 52 can control, based on the monitoring results received by the receiving module 522, the sound module 521 to be switched on via the switch module 523, so as to play the preset sound to the user. After that, the volume tuning module 524 can further slowly adjust the volume of sound based on the monitoring results received by the receiving module 522, so as to vary the sound gradually from low to high. Thereby, the user is wakened in a comparatively gentle manner via slow changes in the volume of sound.

Obviously, having benefits from the present disclosure and teaching of the present disclosure, other gentle wake-up systems 12, 42, 52 having an equivalent effect can be easily obtained by those skilled in the art, as long as the user can be wakened in a gentle manner. Exemplarily, in addition to the optical manner and the acoustic manner as listed above, such gentle wake-up systems 12, 42, 52 can be further implemented through a mechanical measure or an olfactory system and the like.

In other specific embodiments, the massage system 13 can comprise a microwave massage system 130 for giving message to the user's eyes via a microwave massage function, as shown in FIG. 1. Exemplarily, the microwave massage system 130 is arranged in the intermediate layer (for example, a layer sandwiched between the internal layer and the external layer in FIGS. 2, 3) of the body 21, 31. Specifically, the microwave massage system 130 comprises one or more microwave generators (not shown) for giving microwave-massage to acupuncture points of the user's eyes. In these embodiments, by giving massage, specifically microwave-massage, to the user's eyes based on the monitoring results, beneficial effects such as relaxation of nerves and improvement of eye fatigue will be achieved.

Obviously, based on the present disclosure and teaching of the present disclosure, those skilled in the art can easily foresee other similar massage systems. Exemplarily, in addition to the massage given to acupuncture points of the eyes, such a massage system can further aim at other acupuncture points or nerve sites in the vicinity of the eyeshield, so as to alleviate fatigue and improve sleep quality.

In further specific embodiments, the smart eyeshield 10 can further comprise: a communication module 14 coupled to the sleep monitoring system 11 and used for providing to an external device 15 information about the sleep state monitored by the sleep monitoring system 11, as shown in FIG. 1. Exemplarily, the communication module 14 is arranged in the external layer (for example, a layer opposite to the layer facing the observer in FIGS. 2, 3) of the body 21, 31. Furthermore, the communication module 14 can further provide the information about the sleep state to the external device 15 via a Bluetooth module or a wireless module 16. Exemplarily, the Bluetooth module and the wireless module 16 are arranged in the external layer (for example, a layer opposite to the layer facing the observer in FIGS. 2, 3) of the body 21, 31. With the above communication module 14, various information about the sleep quality collected by the sleep monitoring system 11 is timely transmitted to an external device 15 such as a smart phone or a tablet computer. This helps to facilitate establishment of a sleep quality log for the user. For the user or furthermore for medical care personnel, such a sleep quality log not only enables an intuitional understanding of an evolutionary trend of the sleep state with time, but also provides a preventive measure which is effective against poor sleep quality.

Obviously, having benefits from the spirit and teaching of the present disclosure, those skilled in the art can make any suitable modification or variations to the smart eyeshield and various components inside, so as to combine equivalent functions and technical effects described therein. As an example, the sleep monitoring system can be implemented by several sensors, and such sensors can be attached to the user's skin at crucial acupuncture points or nerve sites. Specifically, such sensors can even be metal sensors. Of course, the present disclosure is by no means limited thereto.

It should be pointed out that in the above depictions of the present disclosure, specific structures, dimensions, positions and so on of the smart eyeshield and various components inside are not limited only to the situations as described in the embodiments. On the contrary, having benefits from the spirit and teaching of the present disclosure, those skilled in the art can design specific component structures, dimensions, positions and so on which are suitable according to specific situations.

It should be further noted that in depictions of the above disclosure, terms such as “connect” should be understood in a broad sense, unless otherwise prescribed or defined explicitly. In other words, the connection can be a fixed connection, a removable connection or an integral connection. Of course, the connection can also be a direct connection, an indirect connection via an intermediary, or an internal communication between two elements. For a person having ordinary skills in the art, he/she can understand specific meanings of the above terms in the present disclosure upon specific situations.

In depictions of the present disclosure, specific features, structures, materials or characteristics can be combined in any suitable manner according to any one or more embodiments or examples.

What described above is only specific embodiments of the present disclosure, but the protection scope of the present disclosure is not limited only thereto. Any other variation or substitution easily conceivable within the technical field disclosed in the present disclosure for a skilled person who is familiar with this technical field shall fall within the protection scope of the present disclosure. Therefore, the protection scope of the present disclosure shall be subject to the protection scope of the claims.

1. A smart eyeshield, comprising:
a sleep monitoring system for tracking and monitoring a sleep state of a user; and
a gentle wake-up system for wakening the user in a gentle manner depending on a feedback provided by the sleep monitoring system.

2. The smart eyeshield according to claim 1, further comprising:
a massage system for providing a massage to the user based on the feedback provided by the sleep monitoring system.

3. The smart eyeshield according to claim 2, wherein
the smart eyeshield further comprises a body and a fastener, wherein in a wearing state of the smart eyeshield, the body covers the user's eyes and the fastener surrounds the user's head, and
the sleep monitoring system, the gentle wake-up system and the massage system are arranged within the body.

4. The smart eyeshield according to claim 3, wherein
the body comprises an external layer, an intermediate layer and an internal layer, wherein in the wearing state of the smart eyeshield, the external layer is exposed to outside, the internal layer is in contact with the user, and the intermediate layer is sandwiched between the external layer and the internal layer.

5. The smart eyeshield according to claim 4, wherein the sleep monitoring system comprises:
an eyeball movement catcher; and
a brain vasculature monitor.

6. The smart eyeshield according to claim 5, wherein
the eyeball movement catcher and the brain vasculature monitor are arranged in the internal layer of the body.

7. The smart eyeshield according to claim 6, wherein the eyeball movement catcher comprises:
one or more sensors for recording a rotation frequency of the user's eyeball.

8. The smart eyeshield according to claim 6, wherein the brain vasculature monitor comprises:
one or more pulse sensors for recording a beating frequency of the user's pulses.

9. The smart eyeshield according to claim 4, wherein the gentle wake-up system comprises:
a light emitting module;
a receiving module coupled to the sleep monitoring system and used for receiving a feedback provided by the sleep monitoring system;
a switch module coupled to the receiving module and used for controlling the light emitting module to be switched on or off based on the feedback received by the receiving module; and
a brightness dimming module coupled to the receiving module and used for adjusting brightness of light emitted by the light emitting module based on the feedback received by the receiving module.

10. The smart eyeshield according to claim 9, wherein
the receiving module, the switch module and the brightness dimming module are arranged in the external layer of the body,
the light emitting module is arranged in the intermediate layer of the body, and
the internal layer of the body is made of a light transmissive material.

11. The smart eyeshield according to claim 4, wherein the gentle wake-up system comprises:
a sound module;
a receiving module coupled to the sleep monitoring system and used for receiving a feedback provided by the sleep monitoring system;
a switch module coupled to the receiving module and used for controlling the sound module to be switched on or off based on the feedback received by the receiving module; and
a volume tuning module coupled to the receiving module and used for adjusting volume of the sound module based on the feedback received by the receiving module.

12. The smart eyeshield according to claim 11, wherein
the receiving module, the switch module and the volume tuning module are arranged in the external layer of the body, and
the sound module is arranged in the intermediate layer of the body.

13. The smart eyeshield according to claim 4, wherein the massage system comprises:
a microwave massage system for giving massage to the user's eyes via a microwave massage function.

14. The smart eyeshield according to claim 13, wherein the microwave massage system is arranged in the intermediate layer of the body.

15. The smart eyeshield according to claim 14, wherein the massage system comprises:
one or more microwave generators for giving microwave-massage to acupuncture points of the user's eyes.

16. The smart eyeshield according to claim 4, further comprising:
a communication module coupled to the sleep monitoring system and used for providing to an external device information about the sleep state monitored by the sleep monitoring system.

17. The smart eyeshield according to claim 16, wherein
the communication module is arranged in the external layer of the body.
